(19) United States
(12) Patent Application Publication
(10) Pub. No.: US 2022/0392631 A1
(43) Pub. Date: Dec. 8, 2022

(54) MEDICAL DEVICE WITH A DATA TRANSFER SYSTEM AND METHOD FOR ENSURING THE COMPLETENESS OF A DATA STREAM

(71) Applicant: B. Braun Melsungen AG, Melsungen (DE)

(72) Inventor: Horst Schmoll, Guxhagen (DE)

(21) Appl. No.: 17/775,987

(22) PCT Filed: Nov. 11, 2020
(86) PCT No.: PCT/EP2020/081800

§ 371 Date: May 11, 2022

(30) Foreign Application Priority Data
Nov. 12, 2019 (DE) 10 2019 130 410.5

(51) Int. Cl. G16H 40/67; H04L 1/00; H04L 43/0823

(52) U.S. Cl. CPC G16H 40/67; H04L 1/0083; H04L 43/0823; A61M 5/172

(57) ABSTRACT

A medical device includes a data transfer system with a transmitter device designed to send a data stream. At least one receiver device receives the data stream, and at least one transfer device transfers the data stream from the transmitter device to the receiver device. The transmitter device divides the data stream into individual data packets with a predetermined format. The receiver device includes a completeness checking mechanism that receives the individual data packets in the predetermined format and ensures the completeness of the data stream.

### CROSS-REFERENCE TO RELATED APPLICATIONS

This application is the United States national stage entry of International Application No. PCT/EP2020/081800, filed Nov. 11, 2020, and claims priority to German Application No. 10 2019 130 410.5, filed Nov. 12, 2019. The contents of International Application No. PCT/EP2020/081800 and German Application No. 10 2019 130 410.5 are incorporated by reference herein in their entireties.

### FIELD

The present disclosure relates to a medical device having a data transfer system comprising a transmitter device, preferably an infusion pump or a medical application, configured to transmit a data stream, at least one receiver device, preferably at least one medical application or an infusion pump, configured to receive the data stream, and at least one transfer device configured to transfer the data stream from the transmitter device to the receiver device, and a method for ensuring completeness of a data stream.

### BACKGROUND

In many hospitals worldwide, medical products are connected to an IT network. The focus here is on the exchange of data between medical products and medical applications within the framework of an IT network.

For example, the product marketed by B. Braun under the trademark ONLINESUITE™ implements processes related to infusion therapy, which make it possible to maintain an overview of all infusion pumps in a station, to create and send medication databases from a central location, to generate reports and statistics on the medication applied, and to organize and manage the equipment pool of the infusion pumps. Standardized IT technologies are used for this purpose, which at the same time enable easy integration into existing hospital and IT infrastructure.

There are no known data transmission methods that detect losses in a data stream to be transmitted between the source and the receiver of the data. Due to the fact that in the prior art the integrity and completeness of the received data is not given, the data cannot be used for therapeutic decisions without additional verification. In other words, it is not possible to, for example, make therapeutic decisions or to secure distributed alarm systems solely on the basis of this data. The completeness of a data stream cannot be guaranteed, since the current data transfer systems do not provide any verification options for this.

### SUMMARY

It is therefore the object of the invention to avoid or at least reduce the disadvantages of the prior art. In particular, both integrity and completeness of a data stream are to be ensured.

This object is solved by providing a medical device, in particular infusion pumps or a medical application, with a data transfer system, comprising a transmitter device configured to transmit a data stream, at least one receiver device configured to receive the data stream, and at least one transfer device configured to transfer the data stream from the transmitter device to the receiver device. The transmitter device is preferably a medical device, for example an infusion pump, or a medical application and the receiver device is preferably at least one medical application (software application) or at least one medical device, for example at least one (further) infusion pump.

Here, the transmitter device is provided and configured to split the data stream into individual data packets having a predetermined format, and the receiver device is provided with a completeness checking mechanism that is provided and configured to receive the individual data packets in the predetermined format and to ensure the completeness of the transmitted data stream.

In other words, the predetermined format of the individual data packets is maintained during the entire transfer process. Thus, the predetermined format is not only used for transmission, but also for subsequent storage. In this way, the at least one receiver device is configured to perform a secure single error detection of integrity and completeness of the at least one data stream. Here, the validity of each individual data packet is ensured by an integrity check of the individual data packet.

An integrity check is a check for completeness or intactness. In the present invention, integrity checking is used to check data, in this case divided into data packets, after transmission.

The completeness checking mechanism is provided and configured to check whether the data stream transmitted by the transmitter device, split into the individual data packets/data sets, has arrived completely at the at least one receiver device. The completeness checking mechanism ensures that no data has been lost during sending/transmission. Here, the checking mechanism uses the predetermined format of the individual data packets, which is kept/maintained during the entire transfer process. In other words, this means that the data stream is split into individual data packets, the individual data packets are each packed into a predetermined format and are sent/transmitted in this form from the transmitter device to the receiver device. By maintaining the predetermined format over the entire transmission period, the completeness checking mechanism verifies whether all data packets have arrived, and thus whether the data stream is whole/complete.

This has the advantage that it can be ensured that all data (data stream) reported/to be transmitted by at least one medical product or application is transferred to at least one other medical product or at least one other application and has been received by the at least one other medical product or the at least one other application. In this way, it is possible to detect the completeness of the data stream, i.e. of all data (packets), or respectively to detect data loss. In other words, it is possible to detect whether all data (packets) of a data stream have been transferred completely. The first-error-proof data exchange enables the use of data in server applications for therapeutic decisions, medical alarm management (primary alarm systems), remote control of medical products as well as closed-loop applications using the data of single devices or a multitude of medical products for remote control of medical products, for example Glucose Control, TCI, etc.).

The first-error-proof data transmission and storage enable the development of new data technology applications up to closed-loop systems and the remote control of medical devices. Here it is particularly important to be able to guarantee that data packets and data streams are transferred completely and that the successful and complete transfer can also be checked independently, both immediately after the transfer and at a significantly later point in time, e.g. after a few days, months, or years.

Furthermore, the data transfer system enables medical data applications that, for example, write reports on the basis of which therapeutic decisions can be made.

Advantageous embodiments are explained in more detail below.

It is further preferred if the transmitter device is provided and configured to generate events and to generate a corresponding data packet for each generated event. It is furthermore preferred if the transmitter device generates discontinuous events. For example, the events are triggered by changes in the state of the transmitter device, for example the medical product. Although the timely occurrence of reported events is not specified, each data packet is generated according to the following aspects.

It is preferred if each data packet is assigned the following information. A data packet is defined by the fact that it has a time stamp that determines the time at which the event occurred. In addition, the data packet has an information element that is configured to unambiguously identify individual data packets in a data stream. It is preferred if the information element is a sequential number. Furthermore, each data packet has a live load that comprises/has stored information, for example the medication that is related to the generated event. In order to check the completeness of each data packet, an integrity test is performed, which is done using a check sum of the live load and the time stamp data. In other words, each data packet has a further element for performing the integrity test, which is formed from a check sum of the live load and the data of the time stamp.

In other words, the completeness of the data stream is ensured by checking the data transfer from data split by the transmitter device into sequential data packets via the transfer device to the receiver device. The data packets are provided in a predetermined order when they are split. Based on this order, the present data transfer system makes it possible to detect the completeness of the data stream by checking the data packets at the receiver device.

While the validity of a single data packet is ensured by an integrity check of the individual packet, the completeness of the data stream is ensured by sequential information elements contained in each data packet. Thus, this transmission system allows each receiver device of the application to check the integrity of an individual data packet as well as the completeness of the data stream. In this case, end-to-end monitoring of the sequence of data packets from the source/origin to the processing of the messages in the at least one and each receiver device is applied/performed.

It is preferred if the predetermined format of each data packet for transfer of the data stream is defined by the time stamp, the live load, and the information element, the data stream is formed by several sequential information elements, each of which is provided in a data packet, and the data packets are stored in a preferably non-volatile memory unit. Furthermore, it is preferred if the information elements are configured as consecutive numbers or letters and in this way determine the order of the data packets. In other words, both the predetermined format and the corresponding information elements are stored in the memory unit during the entire transfer process so that they can be retrieved at any time, even in the event of a power failure.

In other words, the first data packet generated is assigned the number ‘1’ or the letter ‘A’, for example, and the second data packet generated is assigned the consecutive number ‘2’ or the consecutive letter ‘B’. For further data packets this principle is continued. Thus, the transmitter device defines an order which enables the at least one receiver device to check the completeness of the data packets based on the information elements. In order to be able to check whether the last data packet of a data stream is missing or has arrived at the receiver device, the information about the number of data packets can, for example, be sent to the receiver device in advance, or alternatively the information element has at least two further digits in addition to the elements listed above, wherein the first digit is a consecutive number which designates the current packet, and the second digit remains the same for a complete data stream and represents the total number of data packets.

According to a further alternative or additional further development, the start and end information element may be marked/identified separately so that the receiver device recognizes whether another data packet is still to be received or whether the data stream has already been completely transferred.

It is preferred if the transmitter device, the at least one transfer device and the at least one receiver device are provided and configured to check and ensure the integrity of each data packet via an integrity test.

It is preferred if the at least one receiver device is provided and configured to ensure single error detection of the integrity of the respective data packet and the completeness of the corresponding data stream for a predetermined time span, preferably varying in a range from seconds to weeks and/or years. In other words, this means that the time span may vary between short time spans/periods, preferably in a range of seconds, to long time spans/periods, preferably weeks or years. In other words, this means that the predetermined time span is a time span dictated by external regulations, such as some kind of retention policy. That means that the first generated event defines the beginning of the predetermined time span and the last generated event defines the end of the predetermined time span.

It is preferred if the transfer device is designed as a wired or as a wireless transfer device and/or as a memory unit.

Furthermore, it is preferred if the data transfer, in particular a first-error-proof data transfer, and storage are provided and configured to transfer data packets and data streams completely, and to check the successful data transfer directly after the data transfer and/or at a later time, in particular after some days, months or years.

Furthermore, the present invention relates to a method for ensuring completeness of a data stream during a data exchange between a transmitter device and a receiver device via a transfer device comprising the following steps. First, at least one event is generated in the transmitter device by the transmitter device. In other words, this means that the transmitter device generates discontinuous events, which are triggered, for example, by changes in the state of the medical product. For each generated event, a corresponding data packet is created in a further step. Although the timely occurrence of reported events is not specified, each data packet is generated with the following information.

A time stamp is generated for each created data packet. The time stamp defines the time at which the event occurred. In a further step, an information element is generated for each created data packet to unambiguously identify the individual data packet in a data stream. It is preferred if the information elements are sequential numbers or letters or something similar. In other words, the individual data packets are numbered in the order in which the events occurred. Thus, the information elements allow to identify a single data packet in a data stream using the sequential numbers or letters or the like, whereby a fixed order is/will be given. In a final step, the live load is generated, which contains all information/data belonging to the respective event generated, such as a medication.

It is preferred if the information element in the transmitter device is generated according to the following rule

CNR_{new}=CNRₚᵣₑᵥ+1

for keeping the order in case of a failure. The receiver device applies the same rule during a predetermined time span in order to check the completeness of the data stream. Here, ‘CNR’ stands for the information element. In words, the rule describes that the first information element corresponding to the first data packet according to the first event is assigned a digit/number, for example ‘1’, or a letter, for example ‘A’. The following second event with the corresponding data packet is assigned a number or a letter (‘2’ or ‘B’) which is higher by one than that of the first information element. This rule is applied to all further data packets to be created.

Furthermore, it is preferred if the information associated with each event is/gets stored in a preferably non-volatile memory unit. In other words, this means that the information consisting of time stamp, information element, and live load is stored at any time of the transfer process. Thus, the order of the individual data packets can be retrieved/kept even in case of equipment and/or power failure.

Furthermore, it is preferred if the aforementioned information defines a message format/predetermined format for each generated data packet. In other words, the predetermined format consists of the live load, the time stamp, the number of information/data packets and the information elements.

It is preferred if the data packets, as described above, are transmitted using conventional transfer devices. For this purpose, controller routing devices/ controller guide devices, wired and/or wireless transfer devices, but also storage devices may be used. In any case, it is preferred if the format described above is transferred, subjected to an integrity test and/or stored.

This method offers the advantage that the at least one receiver device detects possible data loss by applying the rule CNR_{new}=CNRₚᵣₑᵥ+1. Furthermore, the completeness of a data stream can be determined for a certain time span by applying the same rule to a certain number of events. This means that a time interval starts and the data packets are provided with an information element and a time stamp according to a rule that is constant for each data packet until the end of the time interval has occurred. For a newly starting time span, a different rule may be used to define the information element, which in turn is applied until its end. An event is identified as follows:

Start of a time interval <=time stamp<= end of a time interval.

Furthermore, it is preferred if the method is performed using the data transfer system according to one of the preceding aspects.

Thus, end-to-end completeness of a data stream on the receiver device side can be ensured by this method described above. This method is applied at the origin of the data, according to the transmitter device, for example in a medical product, and is maintained during transport/ transfer and storage of the data stream. The method enables each receiver device of the application to check/ensure the integrity of an individual data packet and also the completeness of the data stream.

### BRIEF DESCRIPTION OF THE DRAWING FIGURES

The invention is explained in more detail below based on a preferred embodiment with reference to the accompanying figures.

FIG. 1 is a representation of the components of the data transfer system; and

FIG. 2 is a flowchart of the method for ensuring completeness of a data stream.

### DETAILED DESCRIPTION

In the following, an embodiment of the present disclosure is described based on the accompanying figures. The figures are merely schematic in nature and are provided for the purpose of understanding the invention. Identical elements are designated by the same reference signs.

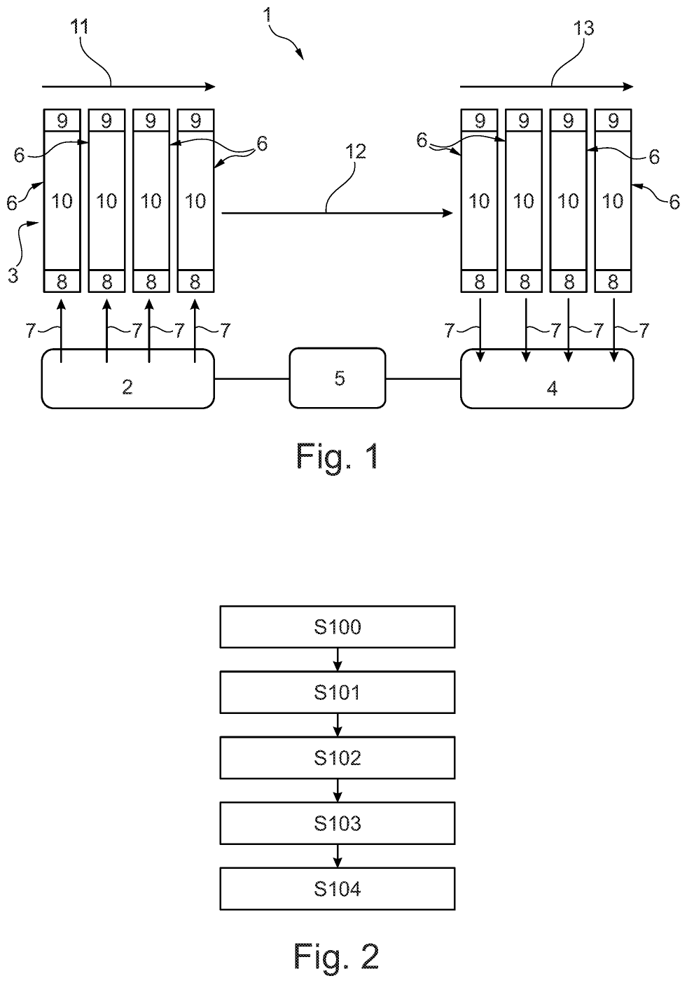

FIG. 1 is a representation of the components of the data transfer system 1. The data transfer system 1 has a transmitter device 2, which is configured to transmit a data stream 3. Furthermore, the data transfer system 1 has a receiver device 4, which is configured to receive the data stream 3. The data transfer system 1 also has a transfer device 5 that is configured to transfer the data stream 3 from the transmitter device 2 to the receiver device 4.

FIG. 1 shows the data stream 3, which is split by the transmitter device 2 into individual data packets 6. In FIG. 1, four data packets 6 are shown on the side of the transmitter device 2, hereinafter referred to as the transmitter side, as well as on the side of the receiver device 4, hereinafter referred to as the receiver side. The four data packets 6 on the transmitter side together form the complete data stream 3 over a certain time span, which is transmitted/transferred via the transfer device 5 to the receiver side to the receiver device 4. The four data packets 6 on the receiver side correspond to the four data packets 6 on the transmitter side and together also form the complete data stream 3.

FIG. 1 shows the events 7 generated by the transmitter device 2. An event 7 is generated, for example, with a change of state of the transmitter device 2. A corresponding data packet 6 is shown for each generated event 7. Each individual data packet 6 has a predetermined format/message format. The predetermined format is defined by a time stamp 8, an information element 9, an integrity test 14 and a live load 10, as well as the number of events 7 or data packets 6, respectively.

The time stamp 8 determines the time at which the event 7 occurred. The information element 10 serves to mark the individual data packets 6 so that they can be clearly identified in a data stream 3. The live load 10 contains all information about the generated event 7. The order of the data packets 6 is defined by the time stamp 8 and the information element 9 as a function of time according to the time's arrow 11 in FIG. 1. Thus, the time span between the first event 7 and the last event 7 of a complete data stream 3 defines the predetermined time span. The integrity test 14 results from a check sum of the live load 10 and the data of the time stamp 8 and serves to ensure the completeness of a data packet 6.

The transfer arrow 12 in FIG. 1 represents the transfer of data packets 6 in the data transfer system 1 from the transmitter device 2 via the transfer device 5 to the receiver device 4.

The receiver device 4 receives the data packets 6 after a transfer. The format of the received data packets 6 corresponds to the format of the data packets 6 transmitted by the transmitter device 2 and is/was maintained during the entire transfer process. The receiver device 4 receives the data packets 6 in the same order depending on the time according to the time's arrow 13 on the receiver side in which the data packets 6 were transmitted by the transmitter device 2. By maintaining the order and based on the information element 9, the verification of the completeness of the data stream 3 from receiver device 4 is possible.

If the number of transmitted data packets 6 corresponds to the number of received data packets 6 and their orders based on the information element, and the integrity check of the individual data packets 6 was successful, the data stream 3 has completely arrived at the receiver device 4.

The receiver device 4 processes the received data packets 6 and the events 7. During the entire transfer process, the data packets 6 are stored in a preferably non-volatile memory unit (not shown). The memory unit is located in the transmitter device. Such a memory unit has the advantage that during the entire transfer process a backup is available, which can be used in case of an incomplete transfer or respectively a data loss during the transfer.

FIG. 2 is a flowchart of the method for ensuring completeness of a data stream 3. The method for ensuring completeness of a data stream 3 during a data exchange between a transmitter device 2 and a receiver device 4 via a transfer device 5 is performed with the following steps.

In a step S100, at least one event 7 is first generated in the transmitter device 2 by the transmitter device 2. In a next step S101, a corresponding data packet 6, which is provided with a predetermined format, is created for each generated event 7.

In accordance with the predetermined format, a time stamp 8, an information element 9 for unambiguous identification of the individual data packet 6 in a data stream 3 and a live load 10 are generated for each created data packet 6, said live load 10 comprising all information/data belonging to the respectively generated event 7. In other words, data of a data stream 3 is acquired and the live load 10 is split among different data packets 6, wherein the individual data packets 6 are in turn provided with additional information. The entire data packet 6 consisting of live load 10, time stamp 8, and information element 9 is subjected to an integrity test 14 and stored on the receiving unit in order to be able to verify the integrity of the individual data packets 6 and the completeness of the data stream 3 immediately but also after a time span defined by e.g. regulatory requirements after a transfer.

As an exemplary information element 9 of the data packets 6, for example, the sequential numbers 1 to 4 or the letters A to D may be provided to define an order which can be followed by the receiver device 4.

In a step S102, the data packets 6 are transmitted via the transfer device 5. Here, the predetermined format is maintained and the data packets 6 are stored during the entire transfer process. In a last step S103 the data packets 6 are received by the receiver device 4. The individual data packets 6 are subjected to an integrity check and the data stream 3 is checked for completeness based on the assigned information elements 9.

1.-11. (canceled)

12. A medical device having a data transfer system comprising:
a transmitter device configured to transmit a data stream;
at least one receiver device configured to receive the data stream; and
at least one transfer device configured to transfer the data stream from the transmitter device to the at least one receiver device,
the transmitter device configured to split the data stream into data packets having a predetermined format, and the at least one receiver device being provided with a completeness checking mechanism configured to receive the data packets in the predetermined format and to ensure completeness of the data stream,
the transmitter device also configured to generate a plurality of events, with each data packet associated with a corresponding event of the plurality of events.

13. The medical device having a data transfer system according to claim 12, wherein each data packet is generated with the following information:
a time stamp that determines a time at which the corresponding event occurred;
an information element configured to unambiguously identify the data packets in the data stream; and
a live load that comprises all information about the corresponding event.

14. The medical device having a data transfer system according to claim 13,
wherein the predetermined format of each data packet for transfer of the data stream is defined by the time stamp, the live load, and the information element, the data stream being formed by a plurality of sequential information elements,
wherein each of the plurality of sequential information elements is provided in one of the data packets, and
wherein the data packets are stored in a memory unit.

15. The medical device having a data transfer system according to claim 12, wherein the transmitter device, the at least one transfer device, and the at least one receiver device are configured to check and ensure integrity of each data packet.

16. The medical device having a data transfer system according to claim 12, wherein the at least one receiver device is configured to ensure single error detection of the integrity of each data packet and the completeness of the data stream for a predetermined time span.

17. The medical device having a data transfer system according to claim 12, wherein the at least one transfer device is a wired transfer device, a wireless transfer device, or a memory unit.

18. The medical device having data transfer system according to claim 12, wherein a data transfer and a data storage are provided and configured to transfer each data packet and the data stream completely, and to check that the data transfer is successful immediately after said data transfer and/or at a later time.

19. A method for ensuring completeness of a data stream during a data exchange between a transmitter device and a receiver device via a transfer device, comprising the following steps:
generating events by the transmitter device and creating data packets corresponding to the events;
generating a time stamp for each data packet;
generating an information element for each data packet to unambiguously identify each data packet in the data stream; and
generating a live load with all information about each event.

20. The method according to claim 19, wherein the information element in the transmitter device is generated according to a rule CNR_{new}=CNRₚᵣₑᵥ+1,
wherein the information element is stored in a memory unit to keep an order in case of a failure as well as to check for completeness of the data stream after the failure, and
wherein the receiver device applies the rule during a predetermined time span to check for completeness of the data stream.

21. The method according to claim 19, wherein the method is performed with a data transfer system comprising:
the transmitter device, which is configured to transmit the data stream;
the receiver device, which is configured to receive the data stream; and
the transfer device, which is configured to transfer the data stream from the transmitter device to the receiver device,
wherein the transmitter device is configured to split the data stream into the data packets having a predetermined format,
wherein the receiver device is provided with a completeness checking mechanism configured to receive the data packets in the predetermined format and to ensure completeness of the data stream, and
wherein the transmitter device is provided and configured to generate events and to create a corresponding data packet for each generated event.
